(19) United States
(12) Patent Application Publication
(10) Pub. No.: US 2025/0000609 A1
(43) Pub. Date: Jan. 2, 2025

(54) RAIL EXTENSION FOR ROBOTIC-SURGERY DEVICES

(71) Applicant: MOMENTIS SURGICAL LTD., Or-Yehuda (IL)

(72) Inventors: Dvir COHEN, Ramot-Menashe (IL); Yaron LEVINSON, Tel Aviv (IL); Gal NACHSHONY, Tel Aviv (IL); Adam RON, Tel Aviv (IL)

(21) Appl. No.: 18/697,751

(22) PCT Filed: Oct. 11, 2022
(86) PCT No.: PCT/IB2022/059717

§ 371 Date: Apr. 2, 2024

Related U.S. Application Data
(60) Provisional application No. 63/262,353, filed on Oct. 11, 2021.

(51) Int. Cl. A61B 90/57

(52) U.S. Cl. CPC A61B 90/57; A61B 2090/571

(57) ABSTRACT

An apparatus for linking a surgical instrument with a rigid frame includes a clamping unit comprising (i) a clamp-actuator and (ii) two opposing clamps attached bilaterally to the clamping unit. Either one of the two opposing clamps can be individually effective, to clamp the clamping unit to the rigid frame. A mediating member is provided for mediating between the surgical instrument and the clamping unit, and is proximally engaged with the clamping unit. Force-transfer mechanisms such as, for example, gearing arrangements, and a plurality of mechanical actuators such as, for example, gear-actuators placed to actuate them, are provided for vertically displacing the mediating member relative to the clamping unit and for pivoting the mediating member in intrinsic rotation in a pitch axis.

### CROSS-REFERENCE TO RELATED APPLICATIONS

This patent application claims the benefit of U.S. Provisional Patent Application No. 63/262,353, filed on Oct. 11, 2021, which is incorporated herein by reference in its entirety.

### FIELD OF THE INVENTION

The present invention relates to positioning systems positionable rail extensions for use with robotic-surgery devices and particularly to robotic-surgery devices comprising one or more elongate mechanical arms.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments can be passed through a cannula and an endoscope can be used to provide images of the surgical site.

Surgical instruments have been developed that utilize an end effector (e.g., a surgical tool such as for tissue fusing or cutting, or a measurement tool) for convenience, accuracy, and wellbeing of the subject. In some cases, articulated surgical arms have one or more bending portions which are controlled remotely using various input devices (e.g., hand and foot controls) to ultimately control the location of the end effector and change its orientation with reference to the surgical arm's longitudinal axis.

Motor-control units include gears which mesh with gears of surgical arms for precise control of the arms. Positioning systems in the form of fixation arms and the like can be used to support and interface with the motor-control units and bring the surgical instruments in proximity to target locations for initiating surgical procedures. Existing positioning systems are known to lack mechanisms for positioning and orienting surgical instruments through three-dimensional space while offering unhindered access to positioning and orientation controls.

### SUMMARY OF THE INVENTION

According to embodiments disclosed herein, an apparatus for linking a surgical instrument with a rigid frame comprises: (a) a clamping unit comprising (i) a clamp-actuator and (ii) two opposing clamps attached bilaterally to the clamping unit, each one of the two opposing clamps being individually effective, when actuated by the clamp-actuator, to clamp the clamping unit to the rigid frame; (b) a mediating member for mediating between the surgical instrument and the clamping unit, proximally engaged with the clamping unit; (c) force-transfer mechanisms for vertically displacing the mediating member relative to the clamping unit and for pivoting the mediating member in intrinsic rotation in a pitch axis; and (d) a plurality of mechanical actuators placed to actuate the force-transfer mechanisms.

In some embodiments, the apparatus is used to directly link the surgical instrument to the rigid frame. In some embodiments, the apparatus is used to link a docking interface and/or instrument-docking assembly to the rigid frame, and in this way is used to indirectly link the surgical instrument to the rigid frame.

In some embodiments, the mediating member can be distally engaged with an instrument-docking assembly arranged to rotate intrinsically in a yaw axis about the mediating member. In some embodiments, the instrument-docking assembly can be detachably attachable to the mediating member by a quick-connect mechanism. In some embodiments, the instrument-docking assembly can include a docking interface adapted for securing the surgical instrument thereto. In some embodiments, the instrument-docking assembly can include a docking interface adapted for securing a motor-control unit of a robotic-surgical device.

In some embodiments, the two clamps can be jointly actuatable by a single clamp-actuator.

In some embodiments, the instrument-docking assembly can comprise a plurality of elongate members pivotably linked to each other.

In some embodiments, the rigid frame can comprise a rail of a patient bed.

In some embodiments, the apparatus can additionally comprise the instrument-docking assembly.

A method is disclosed, according to embodiments, of linking a surgical instrument with a rigid frame. The method can comprise: (a) providing an apparatus for linking a surgical instrument with a rigid frame according to any of the embodiments disclosed herein; (b) attaching the clamping unit to the rigid frame using the clamp-actuator to actuate one of the two opposing clamps; distally engaging, with the mediating member, an instrument-docking assembly comprising a docking interface adapted for securing the surgical instrument thereto; and/or securing the surgical instrument to the docking interface.

In some embodiments, the method can additionally comprise, before the attaching: selecting a vertical orientation of the clamping unit from (i) a first vertical orientation in which the clamp-actuator is disposed above the two opposing clamps and (ii) a second vertical orientation in which the clamp-actuator is disposed below the two opposing clamps, and the attaching can be performed in accordance with the selected vertical orientation.

According to embodiments disclosed herein, an apparatus for supporting a surgical instrument comprises: (a) a clamping unit comprising a clamp-actuator and two opposing clamps attached bilaterally to the clamping unit, each one of the two opposing clamps being individually effective, when actuated by the clamp-actuator, to clamp the clamping unit to a rigid frame; (b) first and second gear-actuators in communication with the clamping unit to define, together with the clamp-actuator, a triangular portion of the clamping unit; (c) a mediating member for mediating between the surgical instrument and the clamping unit, proximally engaged with a first vertex of the triangular portion, (d) a first gearing arrangement disposed at the first vertex and actuatable by the first gear-actuator to vertically displace the mediating member relative to the first vertex; and (e) a second gearing arrangement disposed at a second vertex of the triangular portion and actuatable by the second gear-actuator to pivot the mediating member in intrinsic rotation in a pitch axis about the first vertex.

In some embodiments, the mediating member can be distally engaged with an instrument-docking assembly. In some embodiments, the instrument-docking assembly can be arranged to rotate intrinsically in a yaw axis about the mediating member. In some embodiments, the instrument-docking assembly can be detachably attachable to the mediating member by a quick-connect mechanism. In some embodiments, the instrument-docking assembly can include a docking interface adapted for securing the surgical instrument thereto.

In some embodiments, the instrument-docking assembly can include a docking interface adapted for securing a motor-control unit of a robotic-surgical device.

In some embodiments, it can be that (i) the triangular portion forms a polyhedron bounded by two opposing triangular sides and three quadrilateral sides, and/or (ii) a volume-majority of each one of the first and second gear-actuators is disposed between respective planes defined by the two opposing triangular sides.

In some embodiments, the two clamps can be jointly actuatable by a single clamp-actuator.

In some embodiments, it can be that i) the triangular portion forms a polyhedron bounded by two opposing triangular sides and three quadrilateral sides, and (ii) a volume-majority of the single clamp-actuator is disposed between respective planes defined by the two opposing triangular sides.

In some embodiments, the instrument-docking assembly can comprise a plurality of elongate members pivotably linked to each other.

In some embodiments, it can be that when the clamping unit is clamped to the rigid frame, the clamp-actuator does not intersect a vertical plane defined by a vertical support-frame-facing surface of the rigid frame.

In some embodiments, it can be that when the clamping unit is clamped to the rigid frame, the first and second gear-actuators do not intersect a vertical plane defined by a vertical support-frame-facing surface of the rigid frame.

In some embodiments, the rigid frame can comprise a rail of a patient bed.

In some embodiments, the apparatus can additionally comprise the instrument-docking assembly.

A method is disclosed, according to embodiments, of supporting a surgical instrument. The method can comprise: (a) providing an apparatus for supporting a surgical instrument according to any of the embodiments disclosed herein one of claims 11 to 24; (b) attaching the clamping unit to the rigid frame by using the clamp-actuator to actuate at least one of the two opposing clamps; (c) distally engaging, with the mediating member, an instrument-docking assembly comprising a docking interface adapted for securing the surgical instrument thereto; and/or (d) securing the surgical instrument to the docking interface.

In some embodiments, the method can additionally comprise, before the attaching: selecting a vertical orientation of the clamping unit from (i) a first vertical orientation in which the clamp-actuator is disposed above the two opposing clamps and (ii) a second vertical orientation in which the clamp-actuator is disposed below the two opposing clamps, and the attaching can be performed in accordance with the selected vertical orientation.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIGS. 1A, 1B and 1C are, respectively, schematic perspective (FIG. 1A) and end views (FIGS. 1B, 1C) of an apparatus for supporting a surgical instrument and/or linking it to a rigid frame, shown in a first vertical orientation, according to embodiments of the invention.

FIGS. 1D, 1E and IF are, respectively, schematic perspective (FIG. 1D) and end views (FIGS. 1E, IF) of an apparatus for supporting a surgical instrument and/or linking it to a rigid frame, shown in a second vertical orientation, according to embodiments of the invention.

FIGS. 2A and 2B are, respectively, schematic side and end views of an apparatus for supporting a surgical instrument and linking it to a rigid frame, shown in a first vertical orientation, according to embodiments of the invention.

FIGS. 2C and 2D are, respectively, schematic side and end views of an apparatus for supporting a surgical instrument and linking it to a rigid frame, shown in a second vertical orientation, according to embodiments of the invention.

FIGS. 3A, 3B and 3C illustrate arrangements for pivoting, and vertically displacing, a member arranged to mediate between the apparatus and a surgical instrument, according to embodiments of the invention.

FIGS. 4A and 4B are schematic cutaway drawings of details of an apparatus for supporting a surgical instrument and/or linking it to a rigid frame, showing exemplary gearing arrangements according to embodiments of the invention.

FIGS. 5A and 5B are schematic illustrations of interface-docking assemblies according to embodiments of the invention.

FIG. 6A is a schematic illustration of an exemplary robotic-surgery device.

FIG. 6B is a schematic illustration of the robotic-surgery device of FIG. 6A docked with the interface-docking assembly of FIG. 5A.

FIGS. 7A and 7B show the interface-docking assembly of FIG. 5 engaged with the distal end of a mediating member of the apparatus, in respective first and second vertical orientations, according to embodiments of the invention.

FIGS. 8A and 8B are schematic illustrations of a triangular portion of a clamping unit of the apparatus and respective vertices of the triangular portion, according to embodiments of the invention.

FIG. 9 is a schematic illustration of an apparatus for supporting a surgical instrument and/or linking it to a rigid frame, according to embodiments of the invention.

FIGS. 10A and 10B shows a flowchart of method steps for supporting a surgical instrument and/or linking it to a rigid frame, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

Embodiments disclosed herein relate to fixation arms and rail extensions for robotic-surgery devices using one or more surgical mechanical arms, i.e., articulated mechanical arms, using a plurality of different operating modes and/or a plurality of different input devices. A ‘robotic-surgery device’ as used herein means a device having one or more surgical mechanical arms and a motor unit or motor-control unit for housing and controlling the one or more arms. According to embodiments, a fixation arm is provided for linking a robotic-surgery device to a rigid frame to allow orientation of the robotic-surgery device in a space proximate to a patient with at least three and as many as six degrees of freedom. The fixation arm thus acts as a rail extension. The fixed, rigid frame, in some embodiments, comprises a rail of a patient-bed. In other embodiments, the rigid frame comprises a rail affixed to a wall or other fixture within a room designated for performing a surgical operation.

According to embodiments, one or more ergonometric features can be desirable for the rail extension, including, and not exhaustively: an access feature wherein access to a mechanical actuator for clamping the rail extension to the rail is unblocked and unhindered by the rail, patient-bed or wall, even while clamped; a usability feature wherein the rail extension is usable on either side of a patient-bed, e.g., clampable to a rail on either side without loss of full functionality; an additional access feature wherein access to mechanical actuators for positioning and orienting the robotic-surgery device is unblocked and unhindered by the rail, patient-bed or wall, regardless of which side the rail extension is clamped on; an impingement feature wherein impingement of the rail extension apparatus into the space above a patient-bed is minimized; and a construction feature wherein construction of the apparatus is sufficiently lightweight and mechanically robust so as to ensure that positioning and orientation are maintained once set. A further ergonomic feature can be desirable for the rail extension, singly or in combination with any or all of the foregoing features: a feature according to which the rail is deployable in a plurality of alternative orientations, e.g., vertical orientations, wherein one orientation may be more suitable for a first patient positioning option and a second orientation may be more suitable for a second patient positioning option.

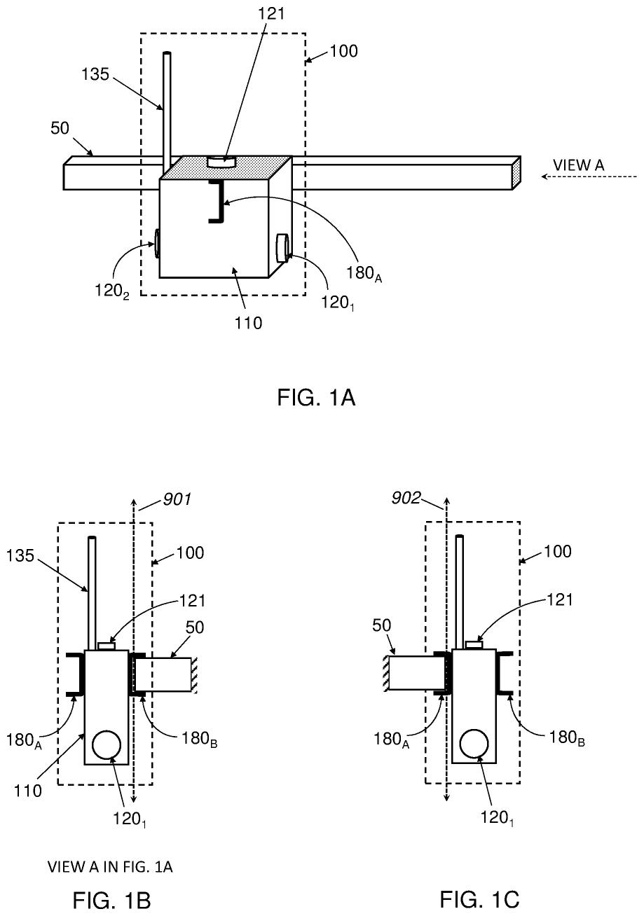

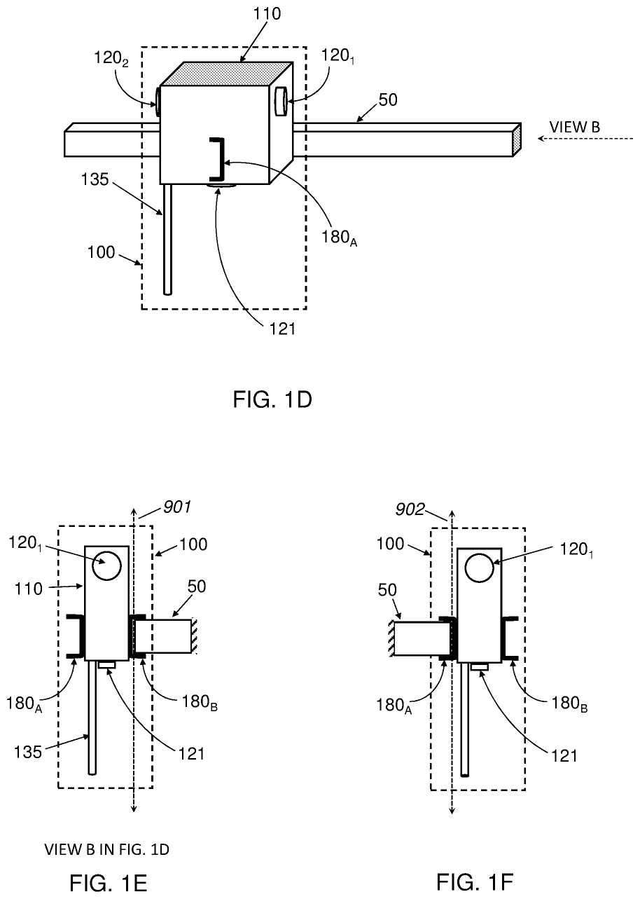

We now refer to the figures and in particular to FIGS. 1A, 1B and 1C.

A rail extension apparatus 100, i.e., an apparatus for supporting a surgical instrument and/or linking it to a rigid frame, includes a clamping unit 110 comprising two clamps 180_{A}, 180_{B} for clamping the apparatus to a rail 50. A clamping unit 100 can have any shape and composition chosen for a specific design. The clamps 180_{A}, 180_{B} are provided bilaterally on the clamping unit 110 and only an ‘A-side’ clamp 180_{A} is visible in FIG. 1A. The corresponding ‘B-side’ clamp 180_{B} is obscured in FIG. 1A by the presence of the clamping unit 110. The clamping unit 110 also includes a mechanical clamp-actuator 121 arranged to actuate the clamps 180_{A}, 180_{B}. In some implementations, as is shown in FIGS. 1A-C, a single clamp-actuator 121 is provided. In other implementations (not shown), there can multiple clamp-actuators 121, e.g., one dedicated to each clamp 180. In the case of a single clamp-actuator 121, the single clamp-actuator 121 is arranged to actuate both clamps 180_{A}, 180_{B} simultaneously, so that it doesn't matter on which side the rail extension 100 is attached to the rail 50. In some embodiments, when the clamping unit 110 is clamped to the rigid frame 50 as shown in FIGS. 1B and 1C, the clamp-actuator 121 does not intersect a vertical plane, respectively indicated by arrows 901 and 902 in FIGS. 1B and 1C, defined by a vertical support-frame-facing surface of the rigid frame 50. FIG. 1B shows an end view (View ‘A’ of FIG. 1A) of the rail extension 100 and rigid frame 50 of FIG. 1A, showing the rail extension 100 clamped to the rigid frame 50 using the ‘B-side’ clamp 180_{B}. FIG. 1C shows a similar end view of the same rail extension 100, clamped to a rigid frame 50 on the ‘other side’, such that the ‘A-side’ clamp 180_{A} is used.

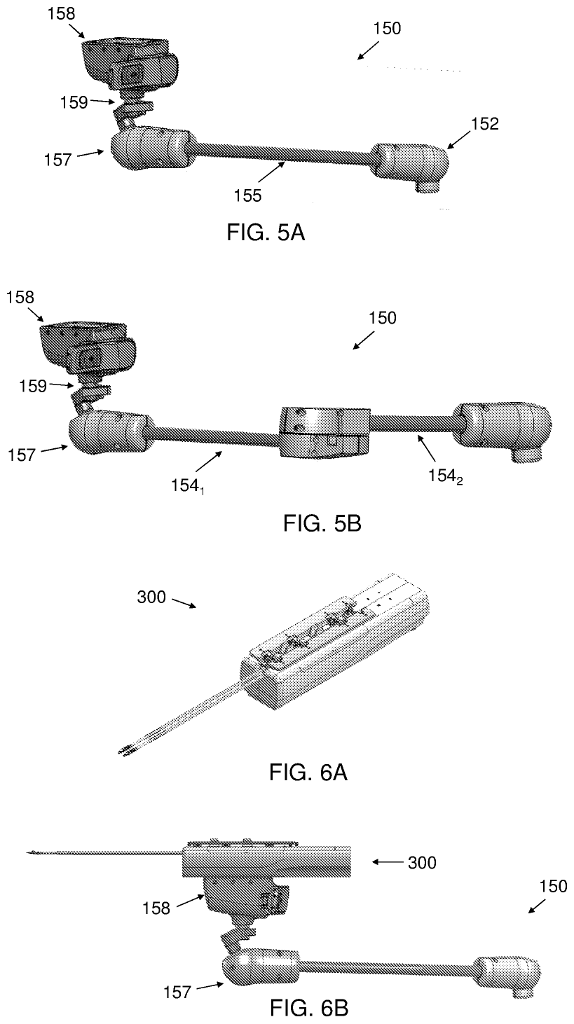

The rail extension 100 also includes a mediating member 135, which is provided to mediate between the clamping unit 110 and the robotic-surgery device (or a docking-interface assembly as will be described in the further description of FIGS. 5-7 hereinbelow). The mediating member 135 is shown throughout the figures as a single narrow rod but in other examples it can include multiple elements joined together, or can be formed as an open frame or truss for increased mechanical robustness with reduced weight.

The rail extension 100 further includes multiple gearing arrangements 125, or, more generally, force-transfer mechanisms, which are illustrated, e.g., in FIGS. 4A-4B. The term “force-transfer mechanism” as used throughout the disclosure and in the claims appended thereto is used to mean any mechanical arrangement or assembly for transferring a force from a “mechanical actuator” to a given element or member of the rail extension 100. A force-transfer mechanism can include one or more gears of any type, and/or one or more threaded elements arranged for force transmission, in any combination. Additionally or alternatively, a force-transfer mechanism can include a non-geared actuation mechanism such as, for example, a non-geared linkage, or a direct and/or integral connection, e.g., of an actuator to or with an actuated member or element. Additionally or alternatively, a force-transfer mechanism can include a mechanism that transfers an actuation force from the actuator to the actuated member using friction. The term “gearing arrangement” as used throughout the disclosure and in the claims appended thereto is used to mean any mechanical arrangement or assembly for transferring a force from a “gear-actuator” to a given element or member of the rail extension 100. A gearing arrangement can include one or more gears of any type, and/or one or more threaded elements arranged for force transmission, in any combination. A gear-actuator is a non-limiting example of a force-transfer mechanism and its use is not intended to exclude non-geared mechanisms. A gear-actuator is a non-limiting example of a mechanical actuator and its use is not intended to exclude actuation of non-geared mechanisms. Thus, the use of the terms “gearing arrangements” and “gear-actuators” can be understood to include any force-transfer mechanisms and corresponding mechanical actuators, respectively, without limitation.

Gear-actuators 120₁, 120₂ are provided for actuating the gearing arrangements 125. Specifically, a first gear actuator 120₁ is arranged to actuate a first gearing arrangement 125₁ (e.g., as shown in FIG. 4B) which is effective to pivot the mediating member 135 in intrinsic rotation in a pitch axis around a pivot point. A second gear actuator 120₂ is arranged to actuate a second gearing arrangement 125₂ (e.g., as shown in FIG. 4A) which is effective to vertically displace the mediating member 135 relative to the clamping unit 110.

The rail extension apparatus 100 is shown in a particular vertical orientation in FIGS. 1A-C, characterized, for example, by the clamp-actuator 121 being higher than the two opposing clamps 180_{A}, 180_{B}. The same rail extension apparatus 100 is shown in a second vertical orientation in FIGS. 1D-F. The second vertical orientation is characterized, for example, by the clamp-actuator 121 being lower than the two opposing clamps 180_{A}, 180_{B}. Notwithstanding the different vertical orientation, the foregoing discussion of FIGS. 1A-C with respect to the structure and functionality of the rail extension apparatus 100 applies as well to FIGS. 1D-F and so is not repeated here. Either of the two vertical orientations can be selected in order to best suit a particular patient position for an operation, such as, for example, and not exhaustively, the Trendelenburg and reverse Trendelenburg positions. For example, the first vertical orientation can be more suitable for a patient positioned in a Trendelenburg position for transvaginal entry to a surgical site. In another example, the second vertical orientation can be more suitable for accessing the lower abdomen of the patient from a higher point, e.g., for making a Pfannenstiel incision.

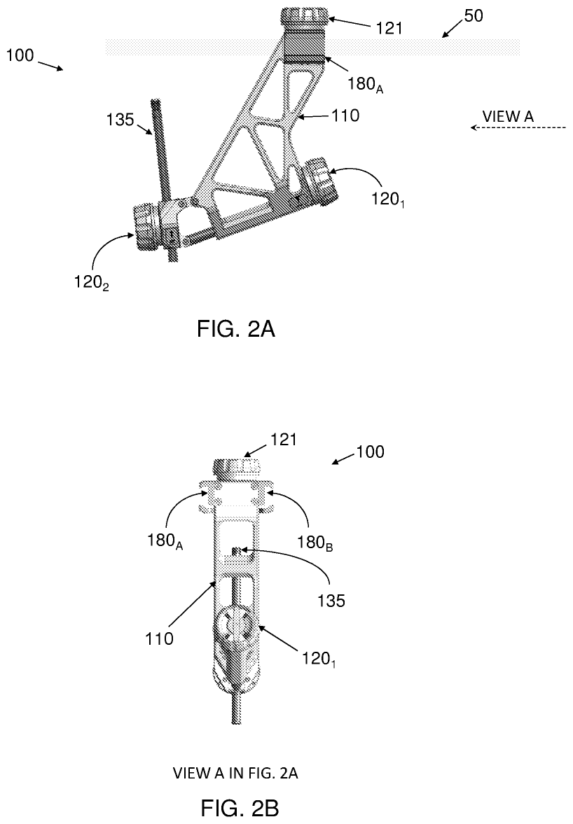

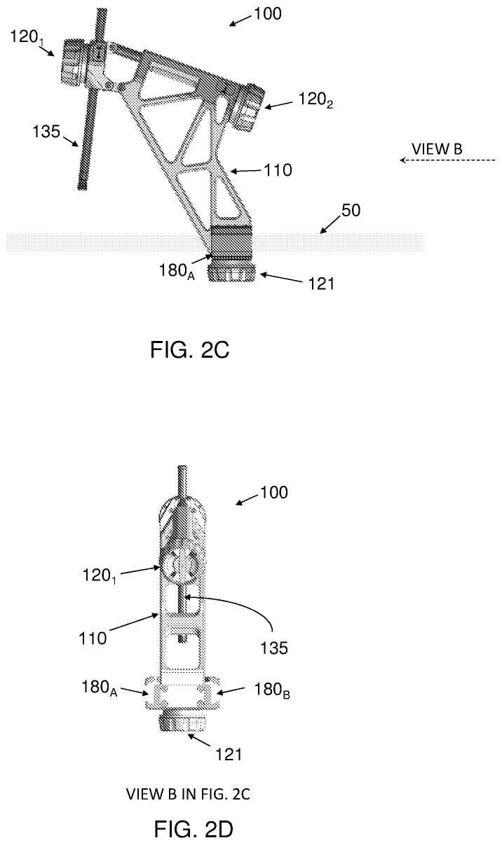

A non-limiting example of a rail extension apparatus 100 is illustrated in FIGS. 2A-2D, where the exemplary rail extension apparatus 100 is shown in a first vertical orientation (that of FIGS. 1A-C) in FIGS. 2A-B, and in a second vertical orientation (that of FIGS. 1D-F) in FIGS. 2C-D. The clamping unit 110 of FIGS. 2A-2D is formed as an open three-dimensional structure designed for mechanical robustness, e.g., resistance to bending moments and reduced weight and/or cost. The rail extension 100 of FIGS. 2A-2D is functionally equivalent to that of FIGS. 1A-IF, and includes the same elements enumerated above: a clamping unit 110 comprising two clamps 180_{A}, 180_{B} and a clamp-actuator arranged to actuate both of the bilaterally-arranged clamps 180, 180_{B}: a mediating member 135 proximally engaged with the clamping unit 110; gearing arrangements 125 for vertically displacing the mediating member 135 relative to the clamping unit 110 and for pivoting the mediating member 135 in intrinsic rotation in a pitch axis; and a plurality of gear-actuators 120 placed to actuate the gearing arrangements 125. The clamps 180_{A}, 180_{B} are shown in FIGS. 2B and 2D in an ‘open’, or unactuated, state. The bed rail 50 is shown in FIGS. 2A and 2C, engaged by the second (obscured) clamp 180_{B}. According to some embodiments, in FIG. 2A, the positioning of the rail extension apparatus 100 relative to the patient-bed is effective to clear the space above the patient-bed, while in FIG. 2C, the positioning of the rail extension apparatus 100 relative to the patient bed is effective to place a robotic-surgery device, e.g., the robotic-surgery device 300 of FIG. 6A, at a higher point relative to the patient.

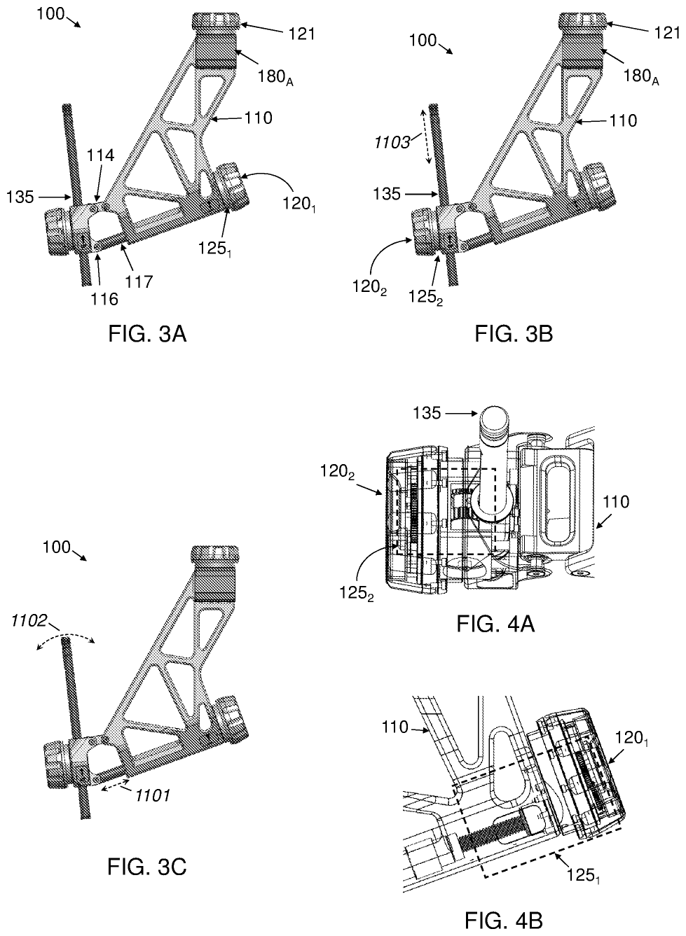

We now refer to FIGS. 3A, 3B, 3C, 4A and 4B.

FIGS. 3A, 3B and 3C schematically illustrate the function of the gearing arrangements 125 and respective gear-actuators 120. The rail extension apparatus 100 is shown in the first vertical orientation of FIGS. 2A-B for convenience, and the disclosed features of, inter alia, FIGS. 3A-C apply equally to rail extension apparatuses 100 deployed in the second vertical orientation of FIGS. 2C-D. The use of the first gearing arrangement 125₁ and the corresponding first gear actuator 120₁ can be understood with reference to FIGS. 3A and 3C. A first gear actuator 120₁ is illustrated as being provided in the form of a knob, as are all of the actuators 120, 121 in the accompanying drawings. In other designs any of the actuators can take other forms such as, for example, levers, switches, or pushbuttons, etc. In a non-limiting example, turning of the first gear actuator 120₁ knob actuates the first gearing arrangement 125₁, which is shown in FIG. 4B as being arranged to convert the radial force of the turning of the knob into longitudinal movement of pushrod 117. The longitudinal movement of pushrod 117 is effective to push or pull pivot element 116 so that the mediating member 135 pivots about pivot arrangement 114 (which can include one or more distinct elements as in the illustrated example). Thus, as seen in FIG. 3C, the longitudinal movement of pushrod 117, indicated by arrow 1101, is effective to cause the mediating member 135 to pivot in intrinsic rotation, i.e., relative to the orientation of the clamping unit 110, in a pitch axis, as indicated by arrow 1102. According to embodiments, any effective mechanism can be employed to effect the pivoting, such as, in a non-limiting example a simpler mechanism involving a push/pull knob as a mechanical actuator rather than a turning knob as described, with the push/pull knob arranged to directly push and pull the pushrod 117 without actual gears or threaded elements.

The use of the second gearing arrangement 125₂ and the corresponding first gear actuator 120₂ can be understood with reference to FIG. 3B. In a non-limiting example, turning of the second gear actuator 120₂ actuates the second gearing arrangement 125₂, which is shown in FIG. 4A as being arranged to convert the radial force of the turning of the knob into longitudinal movement of the mediating member 135, as indicated by arrow 1103. According to embodiments, any effective mechanism can be employed to effect the vertical displacement, such as, in a non-limiting example a simpler mechanism involving a knob affixed to the mediating member 135 and arranged for vertical movement that directly raises and lowers the mediating member 135 without actual gears or threaded elements.

The first and second gear actuators 120₁, 120₂, and the first and second gearing arrangements 125₁, 125₂, can be designed so that the affected movements, i.e., the pitch-axis rotation and vertical displacement, respectively, are precise and determinate. In some embodiments, the design is such that the rotation and/or vertical displacement are actuatable only by the respective actuator(s) 120 and not manually, and/or cessation of an actuation is effective to freeze, or lock, a position or orientation of the mediating member 135 relative to the clamping unit 110. In non-limiting examples, the design can include design elements such as a high-friction component interface, a high gear-reduction ratio, and/or gearing design with reduced or minimal, e.g., zero or close to zero, backlash.

FIGS. 5A and 5B show non-limiting examples of interface-docking assemblies 150 according to embodiments of the invention. An interface-docking assembly is an assembly that includes a docking interface 158 designed to ‘dock’, or interlock, with a robotic-surgery device, e.g., the robotic-surgery device 300 of FIG. 6A. The docking interface 158 includes an upper surface designed to perform the docking with a lower surface of the robotic-surgery device 300. Once a robotic-surgery device 300 is docked with, i.e., secured to, a docking interface 158 as illustrated in FIG. 6B it remains fixed or locked in place until released by a provided release mechanism, e.g., sliding in a specific direction, pressing a button or moving a lever, etc. The interface-docking assemblies 150 of FIGS. 5A and 5B are functionally equivalent in terms of both including a docking interface 158 and a connect mechanism 152 for connection to a mediating member 135. The two interface-docking assemblies 150 differ in that the interface-docking assembly 150 of FIG. 5A includes a single linkage element 155 while the interface-docking assembly 150 of FIG. 5B includes a plurality of elongate members 154₁, 154₂ pivotably linked to each other. The plurality of elongate members 154₁, 154₂ pivotably linked to each other provide an additional method of fine-tuning the orientation of a robotic-surgery device 300, e.g., in a plane defined by the plurality of elongate members 154₁, 154₂. In some embodiments, the docking interface 158 is attached to the linkage elements (e.g., linkage elements 155 or 154) by a ball joint 159 to provide yet another option for fine-tuning the orientation of the robotic-surgery device 300. In the non-limiting examples of FIGS. 5A-B, 6B, and 7A-7B, the ball joint 159 and, with it, the docking interface 158, is supported by a swivel element 157 engaged with the linkage element 155 or elongate member 154.

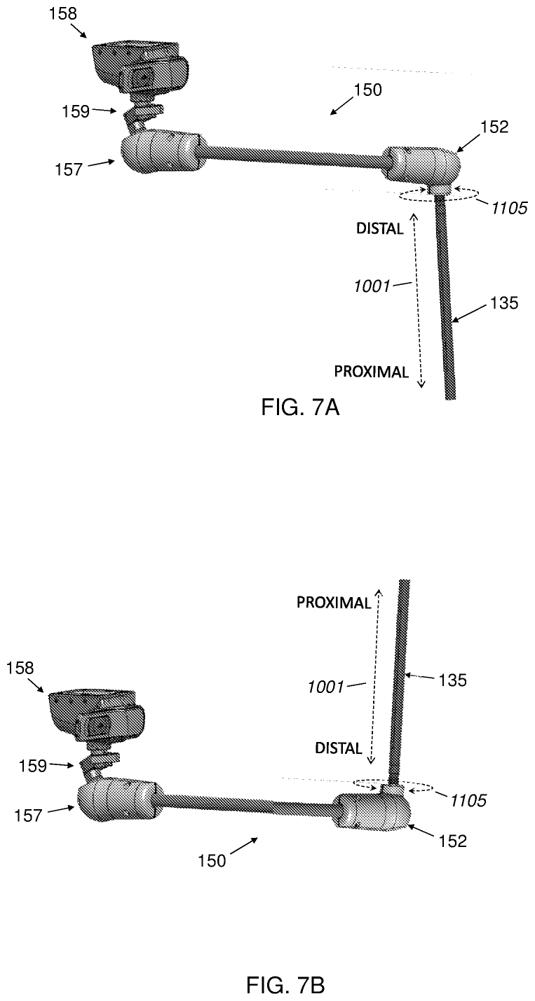

FIGS. 7A and 7B show an exemplary interface-docking assembly 150 connected to the distal end of a mediating member 135 of a rail extension apparatus 100, where the mediating member 135 is the only previously-disclosed part of the rail extension apparatus 100 shown in FIGS. 7A and 7B. The interface-docking assembly 150 of FIG. 7A is arranged for connection to the mediating member 135 of a rail extension apparatus 100 deployed in the first vertical orientation of FIGS. 2A-B, while the interface-docking assembly 150 of FIG. 7B is arranged for connection to the mediating member 135 of a rail extension apparatus 100 deployed in the second vertical orientation of FIGS. 2C-D. In both the first vertical orientation (as shown in FIG. 7A) and the second vertical orientation (as shown in FIG. 7B), the swivel element 157 is arranged such that the ball joint 159 and the docking interface 158 are positioned above the swivel element 157. In some designs, the swivel element 157 is adjustable to enable switching between the first and second vertical orientations while keeping the ball joint 159 and the docking interface 158 above the swivel element 157. In other designs, a different swivel element is used depending on which of the vertical orientations is needed. The term ‘above’ as used here has its usual meaning of vertically higher, and thus the directions ‘above’ and ‘below’ as indicated herein are not dependent on an orientation of any object such as, for example, the rail extension apparatus 100, interface-docking assembly 150, or of a patient-bed.

In some embodiments, the distal end of the mediating member 135 and/or the connect mechanism 152 of the interface-docking assembly 150 is/are adapted for a quick-connect capability, e.g., a snap of the connect mechanism 152 over ridges or protrusions provided at or near the distal end of the mediating member 135. ‘Distal’ as used throughout this disclosure and in the claims appended thereto, with respect to the mediating member 135, means at or toward the end indicated by arrow 1001 in FIG. 7. Once connected, the instrument-docking assembly 150 is arranged to rotate intrinsically in a yaw axis about the mediating member 135, the rotation being that indicated by arrow 1105 in FIG. 7A or FIG. 7B.

In some embodiments, the interface-docking assembly 150 and its components are configured such that any one or more (or all) of the following actions, and not exhaustively, can be performed by a user, e.g., manually, without employing a separate actuator: rotation of the interface-docking assembly 150 in the yaw axis about the mediating member 135; reorientation of elongate members 154₁, 154₂ relative to each other; and reorientation of the robotic-surgery device 300 using the ball joint 159.

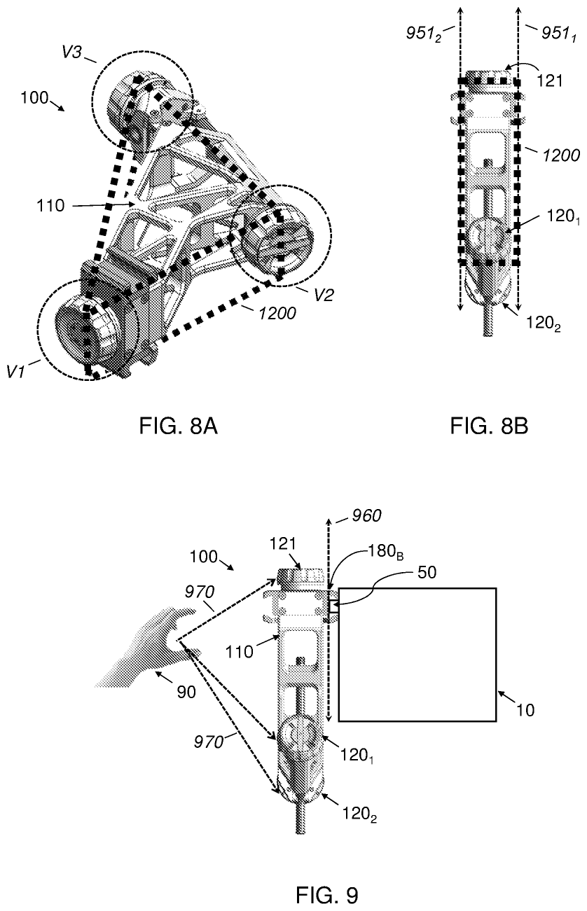

Reference is made to FIGS. 8A and 8B.

The first and second gear-actuators 120₁, 120₂, together with the clamp-actuator 120, define a triangular portion 1200 of the clamping unit 110. The mediating member 135 is engaged with the clamping unit 110 at a first vertex of the triangular portion 1200, marked as V1 in FIG. 8A. The first gearing arrangement 125₁ of FIG. 3A is disposed at the second vertex of the triangular portion 1200, marked as V2 in FIG. 8A. The clamp-actuator 120 is located at the third vertex of the triangular portion 1200, marked as V3 in FIG. 8A. In embodiments, the triangular portion 1200 forms a polyhedron bounded by two opposing triangular sides and three quadrilateral sides. In some designs, the two opposing triangular sides can be parallel or nearly parallel, as in the example of FIG. 8A, making the quadrilateral sides rectangular. In other designs the opposing triangular sides can be non-parallel, i.e., divergent. In some embodiments, such as is shown in the example of FIG. 8B, at least a volume-majority of each one of the first and second gear-actuators 120₁, 120₂ is disposed between respective planes (indicated by arrows 951₁ and 951₂) defined by the two opposing triangular sides. In some embodiments, such as is also shown in the example of FIG. 8B, at least a volume-majority of the clamp-actuator 121 is disposed between the respective planes 951₁ and 951₂ defined by the two opposing triangular sides. An advantage of the gear-actuators 120 and clamp-actuator 121 being disposed between the respective planes 951₁ and 951₂ is that access to the clamp-actuator 121 for clamping and unclamping the rail extension 100 to a rigid frame, e.g., a rail 50, as well as gear-actuators 120 for positioning and orienting the robotic-surgery device 300, are unblocked and unhindered by the rail, patient-bed or wall in any configuration, clamped or unclamped. With the exemplary design illustrated in FIG. 8B, a user is not required to reach under a patient-bed or into a narrow space between the apparatus 100 and a wall in order to actuate any of the mechanical features disclosed in the embodiments. This advantages is illustrated in FIG. 9, where a hand 90 of a user has unhindered access from the ‘A-side’, indicated by the arrows marked 970 to each of the gear-actuators 120 and to the clamp-actuator 121, with the apparatus 100 clamped with the ‘B-side’ clamp 180_{B} to a rail 50 of a patient-bed 10. The hand access in the case of upside-down deployment, i.e., with rail extension apparatus 100 being deployed in the second vertical orientation of FIGS. 2C-D, is the same as that shown in FIG. 9, mutatis mutandis.

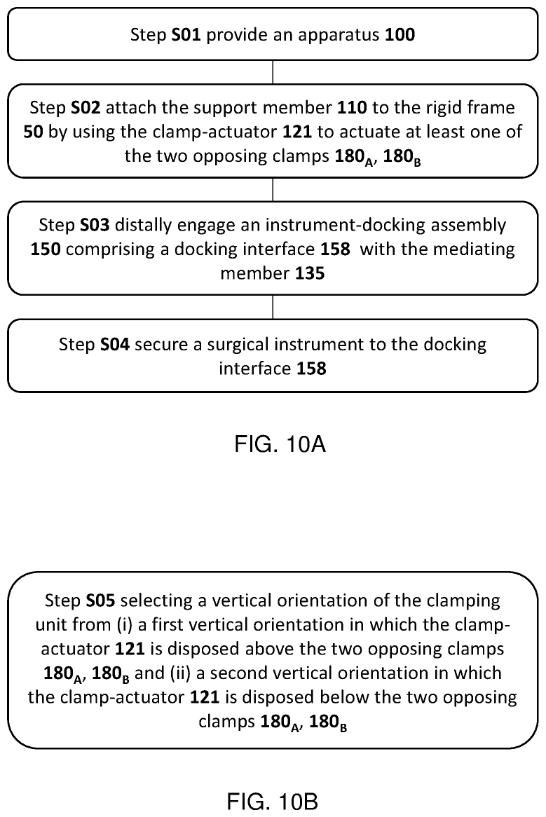

Referring now to FIG. 10A, a method is disclosed for supporting a surgical instrument and/or for linking a surgical instrument with a rigid frame. As illustrated by the flow chart in FIG. 10A, the method comprises:
Step S01 providing a rail extension apparatus 100 in accordance with any of the embodiments disclosed herein.
Step S02 attaching the clamping unit 110 to the rigid frame 50 by using the clamp-actuator 121 to actuate at least one of the two opposing clamps 180_{A}, 180_{B}.
Step S03 distally engaging an instrument-docking assembly 150 comprising a docking interface 158 with the mediating member 135 of the apparatus 100.
Step S04 securing a surgical instrument, e.g., robotic-surgery device 300, to the docking interface 158.

The steps of the method can be performed in any order deemed practical. In some embodiments, not all of the steps are required or performed.

In some embodiments, as shown in FIG. 10B, the method additionally comprises Step S05:
Step S05 includes: selecting a vertical orientation of the clamping unit 110 from (i) a first vertical orientation in which the clamp-actuator 121 is disposed above the two opposing clamps 180_{A}, 180_{B} and (ii) a second vertical orientation in which the clamp-actuator 121 is disposed below the two opposing clamps 180_{A}, 180_{B}. In such embodiments, Step S05 is performed before Step S02, and the attaching of Step S02 is performed in accordance with the vertical orientation selected in Step S05.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. Apparatus for linking a surgical instrument to a rigid frame, the apparatus comprising:
a. a clamping unit comprising (i) a clamp-actuator and (ii) two opposing clamps attached bilaterally to the clamping unit, each one of the two opposing clamps being individually effective, when actuated by a clamp-actuator, to clamp the clamping unit to the rigid frame;
b. a mediating member for mediating between the surgical instrument and the clamping unit, proximally engaged with the clamping unit;
c. respective force-transfer mechanisms for vertically displacing the mediating member relative to the clamping unit and for pivoting the mediating member in intrinsic rotation in a pitch axis; and
d. a plurality of mechanical actuators placed to actuate the force-transmission mechanisms.

2. The apparatus of claim 1, wherein the mediating member is distally engaged with an instrument-docking assembly arranged to rotate intrinsically in a yaw axis about the mediating member.

3. The apparatus of either one of claim 1 or 2, wherein the instrument-docking assembly is detachably attachable to the mediating member by a quick-connect mechanism.

4. The apparatus of any preceding claim, wherein the instrument-docking assembly includes a docking interface adapted for securing the surgical instrument thereto.

5. The apparatus of any one of claims 2 to 4, wherein the instrument-docking assembly includes a docking interface adapted for securing a motor-control unit of a robotic-surgical device.

6. The apparatus of any preceding claim, wherein the two clamps are jointly actuatable by a single clamp-actuator.

7. The apparatus of any one of claims 2 to 6, wherein the instrument-docking assembly comprises a plurality of elongate members pivotably linked to each other.

8. The apparatus of any preceding claim, wherein the rigid frame comprises a rail of a patient bed.

9. The apparatus of any one of claims 2 to 8, additionally comprising the instrument-docking assembly.

10. A method of linking a surgical instrument with a rigid frame, the method comprising:
a. providing an apparatus according to any one of claims 1 to 9;
b. attaching the clamping unit to the rigid frame using the clamp-actuator to actuate one of the two opposing clamps;
c. distally engaging, with the mediating member, an instrument-docking assembly comprising a docking interface adapted for securing the surgical instrument thereto; and
d. securing the surgical instrument to the docking interface.

11. The method of claim 10, additionally comprising, before the attaching: selecting a vertical orientation of the clamping unit from (i) a first vertical orientation in which the clamp-actuator is disposed above the two opposing clamps and (ii) a second vertical orientation in which the clamp-actuator is disposed below the two opposing clamps, wherein the attaching is performed in accordance with the selected vertical orientation.

12. Apparatus for supporting a surgical instrument, the apparatus comprising:
a. a clamping unit comprising a clamp-actuator and two opposing clamps attached bilaterally to the clamping unit, each one of the two opposing clamps being individually effective, when actuated by the clamp-actuator, to clamp the clamping unit to a rigid frame;
b. first and second gear-actuators in communication with the clamping unit to define, together with the clamp-actuator, a triangular portion of the clamping unit;
c. a mediating member for mediating between the surgical instrument and the clamping unit, proximally engaged with a first vertex of the triangular portion,
d. a first gearing arrangement disposed at the first vertex and actuatable by the first gear-actuator to vertically displace the mediating member relative to the first vertex; and
e. a second gearing arrangement disposed at a second vertex of the triangular portion and actuatable by the second gear-actuator to pivot the mediating member in intrinsic rotation in a pitch axis about the first vertex.

13. The apparatus of claim 12, wherein the mediating member is distally engaged with an instrument-docking assembly.

14. The apparatus of claim 13, wherein the instrument-docking assembly is arranged to rotate intrinsically in a yaw axis about the mediating member.

15. The apparatus of either one of claim 13 or 14, wherein the instrument-docking assembly is detachably attachable to the mediating member by a quick-connect mechanism.

16. The apparatus of any one of claims 12 to 15, wherein the instrument-docking assembly includes a docking interface adapted for securing the surgical instrument thereto.

17. The apparatus of any one of claims 12 to 16, wherein the instrument-docking assembly includes a docking interface adapted for securing a motor-control unit of a robotic-surgical device.

18. The apparatus of any one of claims 12 to 17, wherein (i) the triangular portion forms a polyhedron bounded by two opposing triangular sides and three quadrilateral sides, and (ii) a volume-majority of each one of the first and second gear-actuators is disposed between respective planes defined by the two opposing triangular sides.

19. The apparatus of any one of claims 12 to 18, wherein the two clamps are jointly actuatable by a single clamp-actuator.

20. The apparatus of claim 19, wherein i) the triangular portion forms a polyhedron bounded by two opposing triangular sides and three quadrilateral sides, and (ii) a volume-majority of the single clamp-actuator is disposed between respective planes defined by the two opposing triangular sides.

21. The apparatus of any one of claims 13 to 20, wherein the instrument-docking assembly comprises a plurality of elongate members pivotably linked to each other.

22. The apparatus of any one of claims 12 to 21, wherein when the clamping unit is clamped to the rigid frame, the clamp-actuator does not intersect a vertical plane defined by a vertical support-frame-facing surface of the rigid frame.

23. The apparatus of any one of claims 12 to 22, wherein when the clamping unit is clamped to the rigid frame, the first and second gear-actuators do not intersect a vertical plane defined by a vertical support-frame-facing surface of the rigid frame.

24. The apparatus of any one of claims 12 to 23, wherein the rigid frame comprises a rail of a patient bed.

25. The apparatus of any one of claims 13 to 24, additionally comprising the instrument-docking assembly.

26. A method of supporting a surgical instrument, the method comprising:
a. providing an apparatus according to any one of claims 12 to 25;
b. attaching the clamping unit to the rigid frame by using the clamp-actuator to actuate at least one of the two opposing clamps;
c. distally engaging, with the mediating member, an instrument-docking assembly comprising a docking interface adapted for securing the surgical instrument thereto; and
d. securing the surgical instrument to the docking interface.

27. The method of claim 26, additionally comprising, before the attaching: selecting a vertical orientation of the clamping unit from (i) a first vertical orientation in which the clamp-actuator is disposed above the two opposing clamps and (ii) a second vertical orientation in which the clamp-actuator is disposed below the two opposing clamps, wherein the attaching is performed in accordance with the selected vertical orientation.
